(19) United States
(12) Patent Application Publication
(10) Pub. No.: US 2025/0288519 A1
(43) Pub. Date: Sep. 18, 2025

(54) COMPOSITIONS WITH IMPROVED SOLUBILITY OF POOR-WATER-SOLUBLE AGENTS AND METHODS OF MAKING

(71) Applicant: Giffin Technologies Inc., Halifax (CA)

(72) Inventor: David GIFFIN, Halifax (CA)

(21) Appl. No.: 18/607,436

(22) Filed: Mar. 16, 2024

(51) Int. Cl. A61K 9/107; A61K 31/00; A61K 31/685

(52) U.S. Cl. CPC A61K 9/107; A61K 31/658; A61K 31/685

(57) ABSTRACT

Disclosed herein are novel compositions for improving bioavailability of poor-water-soluble agents, which can include nutritional supplements and other ingredients as set forth herein. Also described herein are methods of making the composition and apparatuses used to make the compositions.

### BACKGROUND

Nutritional supplements are used widely for health and wellness. For example, hemp seed oil contains naturally occurring omega-3 and omega-6 at a ratio of 1:3, the optimal ratio for heart and brain health; vitamin A is important for eyesight; vitamin D can reduce cancer cell growth and support immune function; vitamin E, carotenoids, and oregano oil are potent antioxidants; vitamin K is beneficial for bone health; fish oil may reduce inflammation and support heart health, mental health, skin health, bone health, and eye health; algal oil can maintain cognitive health and cardiovascular health; hops oil can reduce fatigue and rejuvenate minds; medium chain triglyceride (MCT) oil is used for weight loss and increasing energy levels; phosphatidylserine promotes cognitive function and memory; phosphatidylcholine supports healthy brain, liver, intestinal, heart, skin, and pregnancy, and may enhance athletic performance; curcumin has anti-inflammatory properties and can relieve joint pain naturally; black cumin seed oil is known for lowering cholesterol and supporting a balanced immune function; mushroom extracts can increase energy and focus and support immune health; ashwagandha root extract may help calm the brain, reduce swelling, lower blood pressure, and alter the immune system; dihydromyricetin has been demonstrated to show antioxidative, anti-inflammatory, anticancer, antimicrobial, cell death-mediating, and lipid and glucose metabolism-regulatory activities; and botanical extracts are used to replenish skin.

However, many nutritional supplements have poor water solubility (i.e., less than 1000 mg/L at 25° C.), which has limited the concentration of nutritional supplements achievable in aqueous solutions and related products. This has historically limited their benefits because low water solubility has led to reduced applicability for incorporation of water-soluble nutritional supplements into aqueous products, such as ready-to-drink products. Furthermore, nutritional supplements in an aqueous solution provide improved bioavailability due to the relationship between bioavailability and the amount of nutritional supplements in an aqueous solution at the site of absorption, typically the small intestine.

Therefore, to improve the delivery of nutritional supplements and to fully utilize the benefits of nutritional supplements, there is a need to develop aqueous compositions with poor-water-soluble agents that have improved bioavailability of the poor-water-soluble agents.

In addition, natural flavor oils and color oils have a growing popularity in food, drinks, and nutritional supplements because they are considered healthier than artificial flavor oils and color oils respectively. However, most of natural flavor oils and color oils are poor water soluble, which can result in their uneven distribution in the food, drinks, and nutritional supplements and thus limit their uses. As such, there is need to improve their water solubility so that they can be readily added to food, drinks, and nutritional supplements.

### SUMMARY

The disclosure contained herein describes novel compositions with increased concentrations of lipophilic and/or low-water-solubility agent (collectively referred to as poor-water-soluble agents). Also contemplated by the disclosure are nutritional-supplements-containing compositions having unexpectedly increased bioavailability and as a result, improved health benefits. Further contemplated by the disclosure are compositions containing natural flavor oils and/or color oils that are ready to be used in food, drinks, and nutritional supplements.

Described herein are novel water-miscible compositions, processes of making the compositions, and apparatuses used to make the compositions.

These and other features, aspects, and advantages of the present embodiments will become understood with reference to the following description and appended claims.

### BRIEF DESCRIPTION OF THE DRAWINGS

FIG. 1 shows a schematic flow diagram of a process for producing compositions described herein.

FIG. 2 shows an exemplary process flow diagram and apparatus used in a process for producing compositions described herein.

FIG. 3 shows an exemplary process flow diagram and apparatus used in a process for producing compositions described herein, wherein the process flow diagram comprises a bypass stream.

FIG. 4 shows an exemplary process flow diagram and apparatus used in a process for producing compositions described herein, wherein the apparatus comprises an example of two ultrasonic processing chambers arranged in parallel.

FIG. 5 shows an exemplary process flow diagram and apparatus used in a process for producing compositions described herein, wherein the process flow diagram comprises an example of a process using an ultrasonic processing chamber for preparing the coarse emulsion.

### DETAILED DESCRIPTION

Described herein are novel compositions with increased concentrations of poor-water-soluble agents in aqueous compositions, which can be used as water-miscible compositions.

In certain embodiments, a poor-water soluble agent can comprise lipophilic nutritional supplements. Also contemplated by the disclosure are compositions having unexpectedly increased bioavailability of the poor-water-soluble agent, and as a result, improved health benefits deriving from their administration to a subject, such as a human or other animal (e.g., mammal). In certain embodiments, a poor-water soluble agent can comprise lipophilic flavor oils. In certain embodiments, a poor-water soluble agent can comprise lipophilic color (e.g., color oils). Further, described herein are novel water-miscible compositions comprising one or more poor-water-soluble agents, one or more surfactants, and water. In certain embodiments, the water-miscible compositions described herein can be incorporated into beverages (such as ready-to-drink beverages) and solid foods (such as vegetables and doughs).

As described herein, poor-water-soluble agents can refer to lipids, lipophilic agents, and/or low-water-soluble agents. As defined herein, lipids can be water-insoluble agents. Low-water-soluble agents are defined herein as agents having a water solubility that is less than a certain threshold value of about 1 g/L at 25° C.

Compositions described herein comprise one or more poor-water-soluble agents, one or more surfactants and water. Compositions prepared in accordance with the method described herein can thus allow for unexpectedly improved increases in the concentration of poor-water-soluble agents, and in the case of low-water-soluble agents, increasing the maximum water solubility achieved by low-water-soluble agents to higher than about 1 g/L at 25° C.

Examples of the lipids include, but are not limited to, hydrocarbon agents, fatty acids, fat-soluble vitamins, curcumin, algal oil, fish oil, hemp seed oil, cannabinoids, flax seed oil, hops oil, oregano oil, MCT oil, phospholipids, cholesterol, vegetable oils, butter, ghee, steroids, waxes (such as honey waxes), cheese, carotenoids, flavor oils, melatonin, magnesium oil, and black seed oil. Hydrocarbon agents include, but are not limited to, terpenes such as monoterpenes (e.g., beta-myrcene), sesquiterpenes (e.g., alpha-humulene and beta-caryophyllene), derivatives thereof, and the like. Fatty acids can be saturated fatty acids, unsaturated fatty acids, polysaturated fatty acids, polyunsaturated fatty acids such as omega 3 fatty acids (e.g., alpha-linolenic acid, eicosapentaenoic acid, and docosahexaenoic acid), omega 6 fatty acids (e.g., linoleic acid, arachidonic acid, gamma linoleic, and conjugated linoleic acid), and omega 9 fatty acids (e.g., oleic acid and erucic acid), lipoic acid, and the like. Notably, lipids can be comprised of specific lipophilic agents, such as for example, alpha-linolenic acid, eicosapentaenoic acid, and docosahexaenoic acid that make up omega 3, and can herein individually be referred to as a lipophilic agent. Fat-soluble vitamins can refer to vitamins A, D, E, K and the like, and any combinations thereof. Examples of the phospholipids include, but are not limited to, phosphatidyl serene and phosphatidylcholine. Examples of flavoring oils include, but are not limited to, cinnamon oil, eucalyptus oil, peppermint oil, clove oil, bay oil, thyme oil, lemon oil, lime oil, orange oil, grape oil, and grapefruit oil. An example of magnesium oil is magnesium chloride hexahydrate. Examples of color oils include, but are not limited to, eucalyptus oil and lavender oil.

Examples of low-water-soluble agents include, but are not limited to, flavonoids (e.g., dihydromyricetin, taxifolin, dihydrokaempferol, engeletin, luteolin, apigenin, tangertin, quercetin, kaempferol, myricetin, fisetin, galangin, isorhamnetin, pachypodol, rhamnazin, hesperetin, naringenin, eriodictyol, homoeriodictyol, pyranoflavonols, withanolides, and furanoflavonol), poor-water-soluble dicarboxylic acids, botanical extracts (e.g., mushroom extracts, capsaicin extract (e.g., phenyl capsaicin), and ashwagandha root extract), and the derivatives thereof. Sources of mushroom extracts include, but are not limited to, Reishi, Lion's Mane, Chaga, Shiitake, Maitake, psilocybe cubensis, and the like. Black cumin seed can serve as the source of black seed oil, but the source of black seed oil is not particularly limited.

An extract can refer to an active ingredient obtained from plants by physical (e.g., pressing) or chemical process (e.g., solvent extraction, distillation, and sublimation) and the meaning of extract will be immediately envisaged by one of skill in the art when viewed in context of the claim terms and disclosure contained herein. Extracts can be substantially free of solvent, i.e., resulting in an active ingredient in a concentrated form containing less than 3000 ppm solvent. As used herein, certain oils such as algal oil, fish oil, hemp seed oil, cannabinoids, flax seed oil, hops oil, oregano oil, MCT oil, flavor oil, and black seed oil may contain more than one lipids. As used herein, when an oil contains one or more lipids and/or lipophilic agents, such an oil can be referred to defined as a lipid oil and is counted as one lipid ingredient in a composition described herein. For example, a composition comprising hemp oil will be considered as comprising one lipid and a composition comprising hemp oil and MCT oil will be considered as comprising two lipids. The skilled artisan would immediately envisage the meaning and scope of the term “lipid oil” when viewed in the context of the surrounding claim terms and disclosure contained herein. A lipid composition described herein can comprise one lipid (such as one of vitamin A, vitamin D, vitamin E, vitamin K, hemp seed oil, and MCT oil), two lipids (such as a combination of hemp seed oil and vitamin D), or three (such as a combination of hemp seed oil, vitamin D, and vitamin E), or more.

A total weight percentage of one or more poor-water-soluble agents in a composition described herein may be between about 5 wt % and about 60 wt %, about 10-50 wt %, about 20-50 wt %, about 30-60 wt %, about 40-60 wt %, about 10%-20 wt %, about 15-30 wt %, about 20-40 wt %, greater than about 10 wt %, greater than about 15 wt %, greater than about 20 wt %, greater than about 30 wt %, greater than about 50 wt %, greater than about 55 wt %, about 10 wt %, about 11 wt %, about 12 wt %, about 13 wt %, about 14 wt %, about 15 wt %, about 16 wt %, about 17 wt %, about 18 wt %, about 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, or any percentage in between.

Composition disclosed herein can further comprise one or more surfactants. Embodiments can comprise one, two, three, or more surfactants. Surfactants can refer to agents that can stabilize compositions described herein and increase the water solubility of poor-water-soluble agents as described herein. Examples of surfactants can include, but are not limited to, phospholipids, triterpenoid saponins, gums (e.g., xanthum gum, guar gum, and gellan gum), celluloses (e.g., carboxymethylcellulose and AKA cellulose), brominated vegetable oil, ammonium phosphatide, acetic acid esters, mustard, sodium stearoyl lactylate, sodium phosphates, diacetyl tartaric acid esters (e.g., diacetyl tartaric acid esters of monoglyceride), polyglycerol esters, phosphates, magnesium stearate, monoglycerides of fatty acids, diglycerides of fatty acids, sucrose esters, sucrose fatty acid ester, sucrose acetate isobutyrate, sorbitan monostearate, sucroglycerides, polyglycerol esters of fatty acids, polyglycerol, polyricinoleate, stearoyl lactylates, sorbitan esters, lactic acid esters, dextrin, polysaccharides, glycoproteins, hydrocolloids, and polysorbates. Examples of the phospholipids include, but are not limited to, dipalmitoylphosphatidylcholine, disteroylphosphatidylcholine, diarachidoylphosphatidylcholine, dibehenoylphosphatidylcholine, diphosphatidyl glycerol, phosphatidylcholines, phosphatidylethanolamines, phosphatidylserines, phosphatidylglycerols, sphingomyelin, lecithin, and phosphatidylinositols. In certain embodiments, a composition described herein comprises lecithin as a phospholipid. In certain embodiments, the lecithin is sunflower lecithin. In certain embodiments, quillaia extract containing triterpenoid saponins is used as a surfactant in compositions described herein. Certain embodiments comprise one or more hydrocolloids as the surfactants. Examples of the hydrocolloids include, but are not limited to, gum acacia, modified food starches, apple cider vinegar, xanthan, galactomannans, carrageenan, pectin, cellulose derivatives, and alginates. In certain embodiments, the hydrocolloid is gum acacia. Certain embodiments comprise one or more polysorbates as the surfactants. Examples of the polysorbates include, but are not limited to, polysorbate 20, polysorbate 40, polysorbate 60, polysorbate 65, and polysorbate 80. In certain embodiments, the polysorbate is polysorbate 80. In certain embodiments, the one or more surfactants can comprise lecithin, quillaia extract, gum acacia, a polysorbate, or any combination thereof. In certain embodiments, the one or more surfactants are selected from the group consisting of sunflower lecithin, quillaia extract, gum acacia, and polysorbate 80. In certain embodiments, a first surfactant, a second surfactant, and a third surfactant are selected from the group consisting of sunflower lecithin, qualia extract, gum acacia, and polysorbate 80. In a particular embodiment, a first surfactant is sunflower lecithin, a second surfactant is polysorbate 80, and a third surfactant is gum acacia.

A weight percentage of each of the one or more surfactants in a composition described herein may be between about 0.1 wt % and about 10 wt %, about 5-20 wt %, about 10-30 wt %, about 20-40 wt %, greater than about 10 wt %, greater than about 15 wt %, greater than about 20 wt %, greater than about 30 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2.0 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, about 3.0 wt %, about 3.1 wt %, about 3.2 wt %, about 3.3 wt %, about 3.4 wt %, about 3.5 wt %, about 3.6 wt %, about 3.7 wt %, about 3.8 wt %, about 3.9 wt %, about 4.0 wt %, about 4.1 wt %, about 4.2 wt %, about 4.3 wt %, about 4.4 wt %, about 4.5 wt %, about 4.6 wt %, about 4.7 wt %, about 4.8 wt %, about 4.9 wt %, about 5.0 wt %, about 5.1 wt %, about 5.2 wt %, about 5.3 wt %, about 5.4 wt %, about 5.5 wt %, about 5.6 wt %, about 5.7 wt %, about 5.8 wt %, about 5.9 wt %, about 6.0 wt %, about 6.1 wt %, about 6.2 wt %, about 6.3 wt %, about 6.4 wt %, about 6.5 wt %, about 6.6 wt %, about 6.7 wt %, about 6.8 wt %, about 6.9 wt %, about 7.0 wt %, about 7.1 wt %, about 7.2 wt %, about 7.3 wt %, about 7.4 wt %, about 7.5 wt %, about 7.6 wt %, about 7.7 wt %, about 7.8 wt %, about 7.9 wt %, about 8.0 wt %, about 8.1 wt %, about 8.2 wt %, about 8.3 wt %, about 8.4 wt %, about 8.5 wt %, about 8.6 wt %, about 8.7 wt %, about 8.8 wt %, about 8.9 wt %, about 9.0 wt %, about 9.1 wt %, about 9.2 wt %, about 9.3 wt %, about 9.4 wt %, about 9.5 wt %, about 9.6 wt %, about 9.7 wt %, about 9.8 wt %, about 9.9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt %, about 41 wt %, about 42 wt %, about 43 wt %, about 44 wt %, about 45 wt %, about 46 wt %, about 47 wt %, about 48 wt %, about 49 wt %, about 50 wt %, about 51 wt %, about 52 wt %, about 53 wt %, about 54 wt %, about 55 wt %, about 56 wt %, about 57 wt %, about 58 wt %, about 59 wt %, about 60 wt %, or any range or percentage in between. A composition described here can comprise more than one surfactant where a weight percentage of each surfactant can be equal or different. For example, a composition described herein can comprise about 1 wt %-3 wt % of a first surfactant and about 2 wt %-4 wt % of a second surfactant; about 1 wt %-3 wt % of a first surfactant and about 5 wt %-8 wt % of a second surfactant; about 2 wt %-4 wt % of a first surfactant and about 5 wt %-8 wt % of a second surfactant; about 1 wt %-3 wt % of a first surfactant, about 2 wt %-4 wt % of a second surfactant, and about 5 wt %-8 wt % of a third surfactant.

In certain embodiments, a composition as described herein has a pH value of about 3.0, 3.5, 4.0, 4.5, 5.0, 5.5, 6.0, 6.5, 7, 7.5, 8.0, 8.5, 9.0, or more or any value in between. In certain embodiments, a composition described herein has a pH value ranging between about 3.5 and 5.5. In certain embodiments, a composition described herein has a pH value of about 5.5.

Compositions described herein can comprise one or more poor-water-soluble agents that are suspended in an aqueous solution as, for example as an emulsion, in accord with the description contained herein wherein the one or more poor-water-soluble agents can each have a specific particle size distribution that contributes to the improved water solubility. In various embodiments, this particle size distribution can comprise an average particle size between about 0.1 um to about 20 um, between about 0.5 um to about 10 um, between about 1.0 um to about 4.0 um, between about 1.5 um to about 3.0 um, between about 2.0 um-2.5 um, or any ranges or values therebetween.

Certain embodiments can comprise a poor-water-soluble agent in an aqueous solution wherein the poor-water-soluble agent comprises one or more of a lipid, a low-water-soluble agent, and a lipophilic agent. In such embodiments, the amounts of the lipid, low-water-soluble agent, and/or lipophilic agent are not particularly limited and can be present individually or collectively in the weight percent amounts set forth herein. The skilled artisan would immediately envisage the relative and total amounts of a lipid, low-water-soluble agent, and/or lipophilic agent to be incorporated into the aqueous compositions set forth herein. For example, compositions described herein can comprise a total weight percentage of one or more poor-water-soluble agents (which can be comprised of one or more lipids and one or more low-water-soluble agents in accord with the proportions described herein) of about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt %, about 41 wt %, about 42 wt %, about 43 wt %, about 44 wt %, about 45 wt %, about 46 wt %, about 47 wt %, about 48 wt %, about 49 wt %, about 50 wt %, or any range or percentage in between, one or more low-water-soluble agents at a total weight percentage of about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt %, about 41 wt %, about 42 wt %, about 43 wt %, about 44 wt %, about 45 wt %, about 46 wt %, about 47 wt %, about 48 wt %, about 49 wt %, about 50 wt %, or any range or percentage in between, and/or one or more lipophilic agents at a total weight percentage of about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt %, about 41 wt %, about 42 wt %, about 43 wt %, about 44 wt %, about 45 wt %, about 46 wt %, about 47 wt %, about 48 wt %, about 49 wt %, about 50 wt %, or any range or percentage in between.

Compositions described herein may contain any number of additional components, including one or more water-soluble agents, acids, bases, coloring agents, flavoring agents, preservatives, pH buffering agents, sweetening agents, and the like. Examples of water-soluble agents include, but are not limited to, magnesium glycinate, magnesium sulfate, magnesium citrate, magnesium L-threonate, thiamine, riboflavin, niacin, pantothenic acid, pyridoxine, biotin, folate, cobalamin, Acids and bases that are considered to be “Generally Recognized as Safe” (“GRAS”) under the Food, Drug, and Cosmetic Act (FD&C) can be employed in compositions described herein adjust pH values of compositions described herein. Examples of coloring agents include, but are not limited to, any of the approved certified water-soluble FD&C dyes. Examples of preservatives include, but are not limited to, glycerin, methyl and propylparaben, benzoic add, sodium benzoate and alcohol. Examples of pH buffering agents include, but are not limited to, acetate, sodium citrate, cyclodextrine derivatives, sorbitan monolaurate, triethanolamine odium acetate, and triethanolamine oleate. Examples of sweetening agents include, but are not limited to, glucose, sucrose, sucralose, syrups, lactitol, maltitol, xylitol, alcohol sugars, sorbitol, glycerin and artificial sweetening agents such as saccharin, aspartame, and acesulfame potassium. Examples of acids include, but are not limited to, tartaric acid, salicylic acid, acetic acid, malic acid, folic acid, fumaric acid, lactic acid, and citric acid. Examples of bases include, but are not limited to, calcium carbonate, trisodium phosphate, potassium bicarbonate, ammonium bicarbonate, calcium hydroxide, sodium benzoate, and sodium bicarbonate. In certain embodiments, citric acid is added to adjust pH values of a composition described herein. In certain embodiments, sodium bicarbonate is added to adjust pH values of a composition described herein.

Also described herein are methods of preparing compositions described herein. An exemplary arrangement of method steps is as follows:
(a) mixing one or more surfactants and water;
(b) adding one or more poor-water-soluble agents to the resultant mixture to yield an emulsion;
(c) homogenizing the resultant emulsion to create a coarse emulsion;
(d) ultrasonicating the coarse emulsion to provide a water-miscible composition;
(e) removing impurities from the water-miscible composition; and
(f) pasteurizing the composition.

In certain embodiments, steps (a) and (b) can be rearranged as would be envisaged by the skilled artisan in view of the disclosure and claims set forth herein. For example, one or more poor-water-soluble agents, one or more surfactants and water can be mixed together in a different order so long as the resultant mixture is sufficiently mixed to obtain a desired concentration of the one or more poor-water-soluble agents. In some embodiments, a first poor-water-soluble agent is added to the mixture, which is then circulated through a processing loop containing a mixer and ultrasonicating chamber, and one or more poor-water-soluble agents can be added to the mixture sequentially or simultaneously. Additional components such as acids or bases can also be added into in step (a) and/or step (b) to adjust the solution's pH as desired to aid in solubilizing the one or more poor-water-soluble agents and/or stabilizing final emulsion particles.

Step (c) homogenization can be achieved by a high shear method, such as by using high pressure homogenizer, microfluidizer, ultrasonication or any combinations thereof. High shear homogenization can be conducted at about 1,000-50,000 rotations/minute (RPM) for at least about two minutes. Micro-fluidization can be conducted under conditions that would be immediately envisaged by one of skill in the art in view of the disclosure contained herein. In a particular embodiment, micro-fluidization can be conducted one or more times (e.g., twice) with a flow rate up to about 500 m/s at a minimum pressure of about 3500 PSI and can utilize one or more y or z-type channels in series or parallel to homogenize a mixture as set forth herein. For homogenization, ultrasonication can be conducted under the same conditions utilized for the ultrasonication step described as step (d), or under a lower oscillation frequency and reduced amplitude. For example, and without limitation, a coarse emulsion can be produced as set forth herein using ultrasonication at an oscillation frequency between about 5,000 to about 10,000 Hz, about 10,000 to about 20,000 Hz, about 20,000 to about 30,000 Hz, and any range therebetween.

In certain embodiments, step (d) ultrasonication can comprise ultrasonicating the homogenized emulsion in an ultrasonic processing chamber where the homogenized emulsion is oscillated at a frequency of about 10,000-40,000 Hz, or about 15,000-30,000 Hz, about 45,000-65,000 Hz, or about 60,000-80,000 Hz, greater than about 80,000 Hz, and ranges therebetween. An ultrasonication step can be conducted using an amplitude of about 0.01-0.1 mm, about 0.4-1.0 mm, about 0.5-0.9 mm, greater than about 0.5 mm and ranges therebetween for a predetermined time range. A predetermined time range is decided by the time to form uniformly distributed particles in an aqueous phase where an average particle size is between 0.1 um to about 20 um. As would be understood by the skilled artisan in view of the description contained herein, a predetermined time range can be considered to be a time to minimize the standard deviation of the average particle size of a poor-water-soluble agent contained in an emulsion described herein, which would also reduce or avoid altogether coalescence of emulsified poor-water-soluble agent particles. In some embodiments, a predetermined time range can be between about 20 minutes to about 40 minutes, about 1 hour to about 3 hours, about 2 hours to about 6 hours, about 4 hours to about 10 hours, about 6 hours to about 14 hours, about 10 hours to about 20 hours, about 12 hours to about 24 hours, or greater than about 24 hours, including ranges therebetween. In certain embodiments, the homogenized emulsion is oscillated at a frequency of about 20,000-40,000 Hz and at an amplitude of about 0.2-1 mm for about 20 minutes to 24 hours. In certain embodiments, the homogenized emulsion is oscillated at a frequency of about 20,000 Hz and at an amplitude of about 0.8 mm for about 20 minutes to 24 hours. As a result, about 144-300 KJ/kg of energy travels through the homogenized emulsion, vibrating and breaking large emulsion particles into much smaller, better uniform sized, and stable emulsion particles. An ultrasonic probe can be used to deliver energy. The ultrasonic probe can be made of titanium, ceramic, glass, alloys or piezoelectric high polymers. In certain embodiments, temperatures of the homogenized emulsion before ultrasonication can be controlled by a heat exchanger. In certain embodiments, a temperature of the emulsion can be about 40° C.-65° C. In certain embodiments, temperatures of the emulsion during ultrasonication can be controlled by a heat exchanger. In certain embodiments, an emulsion's temperature in an ultrasonication chamber can be about 40° C.-65° C. during ultrasonication. In certain embodiments, an emulsion's temperature is about 60° C.

The emulsion mixture can pass from the homogenizer to an ultrasonication chamber, where it can be recirculated through the ultrasonication chamber as defined herein for a predetermined amount of time. The total time of the process is considered to start from the time the initial ingredients are introduced into the homogenizer until the emulsion mixture exits the ultrasonication chamber, which is herein defined as a “processing time” (T_{P}). Processing time can be altered by changing the flow rate, time spent in the homogenizer, size of the system, and the number of passes through the ultrasonication loop, which is herein defined as the “pass number,” (PN). Processing time is not particularly limited, and can be about 10 minutes, about 20 minutes, about 30 minutes, about 45 minutes, about 1 hour, about 2 hours, about 3 hours, about 4 hours, about 5 hours, about 6 hours, about 7 hours, about 8 hours or any amount in between.

In certain embodiments, step (d) ultrasonication can further comprise circulating an ultrasonicated emulsion through a loop where the ultrasonic processing chamber so that the emulsion is ultrasonicated at least three times. Circulation flow rates can be about 1 to about 20 kg/min (kg referring to the total weight of the emulsion). For example, circulation flow rates can be about 1.0 kg/min, about 2.0 kg/min, about 3.0 kg/min, about 4.0 kg/min, about 5.0 kg/min, or more.

In an exemplary embodiment, the ratio of PN to the flow rate (X_{kg/min}) is about 3:1, so at a flow rate of about 1 kg/min, the pass number would be 3. Thus, in this exemplary embodiment, for example, at a flow rate of about 1.0 kg/min, the emulsion would be ultrasonicated 3 times, whereas at a flow rate of about 2.0 kg/min, an emulsion would be ultrasonicated 6 times. The ratio of the pass number to the flow rate is not particularly limited and can be about 1:1, 2:1, 3:1, 4:1, 5:1, 6:1, and ratios therebetween.

Systems described herein can have a residence time (R_{T}) and an exposure time (E_{T}), wherein the residence time can be defined as the time an emulsion mixture spends in an ultrasonic processing chamber in a single pass and calculated by multiplying the length of an ultrasonication chamber by the flow rate of the emulsion mixture:

$${R}_{T} = {L}_{US Chamber} \times {X}_{kg / min}$$

Exposure time can be defined as the total time that an emulsion mixture spends in an ultrasonication chamber or ultrasonic loop as those terms are described herein. Exposure time can thus be calculated as the residence time multiplied by the pass number:

$${E}_{T} = PN \times {R}_{T}$$

In certain embodiments, residence time can be about 10 minutes to about 1 hour. Residence time can range between about 10 minutes to about 30 minutes, about 15 minutes to about 45 minutes, about 30 minutes to about an hour, about 5 minutes to about 10 minutes, about 10 minutes to about 15 minutes, about 10 minutes to about 20 minutes, about 20 minutes to about 30 minutes, and ranges therebetween. In certain embodiments, exposure time can be the same as the residence time or greater than the residence time. In certain embodiments, exposure time can range between about 10 minutes to about 30 minutes, about 15 minutes to about 45 minutes, about 30 minutes to about an hour, about 5 minutes to about 10 minutes, about 10 minutes to about 15 minutes, about 10 minutes to about 20 minutes, about 20 minutes to about 30 minutes, about 1 hour to about 5 hours, about 2 hours to about 4 hours, about 6 hours to about 10 hours, about 4 hours to about 8 hours, about 5 hours to about 12 hours, about 8 hours to about 16 hours, about 10 hours to about 20 hours, about 10 hours to about 15 hours, about 12 hours to about 18 hours, about 10 hours to about 24 hours, about 24 hours to about 36 hours, about 36 hours to about 48 hours, and ranges therebetween. In view of the foregoing, the skilled artisan would immediately envisage how to alter the foregoing variables in view of the disclosure contained herein, the examples, and exemplary descriptions of processes contained herein.

Furthermore, a total processing time within the loop (including homogenization and ultrasonication) can be about 1 hour, about 2 hours, about 4 hours, about 8 hours, about 16 hours, about 24 hours, about 48 hours or more or any hours in between. In certain embodiments, a total processing time is about 24 hours. During ultrasonication, temperatures of an emulsion can be controlled by a heat exchanger to reduce the process time and maintain the integrity of the emulsion particles. For example, emulsion temperatures can be controlled at about 40° C. to about 65° C. In certain embodiments, an emulsion temperature is maintained at about 60° C. during ultrasonication.

In certain embodiments, step (e) can be conducted by centrifuging or settling followed by filtration. Centrifuging/settling removes probe particles from cavitation of the ultrasonic probe and filtration removes any impurities from the homogenization, ultrasonication, and circulation process. A high-capacity centrifuge can be used for centrifuging. Alternatively, settling can be done by keeping an emulsion still in a tank without stirring for a period of about 12 to about 48 hours so that probe particles can settle at the bottom of the tank. Filtration can be carried out by using a sanitary cartridge filter or the like.

In step (f), the emulsion can be pasteurized can be conducted to ensure the resultant composition is safe for human consumption. The pasteurization can be carried out at temperatures lower than 100° C. for about between 15 seconds and 30 minutes. For example, the pasteurization can be carried out at 69° C. for 30 minutes, at 80° C. for 25 seconds, or at 83° C. for 15 seconds.

An apparatus used in a preparation process is also described herein. In certain embodiments, an apparatus comprises a homogenization chamber, a pump, at least two valves, at least one heat exchanger, at least one ultrasonic processing chamber, a sedimentation/settling tank, and a filter.

The homogenization chamber, pump, at least one valve, at least one heat exchanger, and at least one ultrasonic processing chamber form a recirculation loop. Ingredients in a composition described herein can be mixed and homogenized in the homogenization tank to form a coarse emulsion, which can then be further processed via ultrasonication in at least one ultrasonic processing chamber. In certain embodiments, at least one ultrasonic processing chamber can comprise two or more ultrasonic processing chambers which forms an ultrasonication loop, where the two or more ultrasonic processing chambers can be arranged in series, parallel, or in a combination thereof. An ultrasonication loop can recirculate within itself, i.e., after a coarse emulsion is transferred to one or more ultrasonic processing chambers, the product stream exiting the two or more ultrasonic processing chambers can recirculate within the ultrasonication loop back through the ultrasonic processing chambers. In the recirculation loop, the product stream, or a portion thereof, leaving the one or more ultrasonic processing chambers can be transferred back through the homogenization chamber and then through the one or more ultrasonic processing chambers until a predetermined number of passes take place to achieve a predetermined concentration of the poor-water-soluble agent. In certain embodiments, a proportion of the production stream exiting the ultrasonic processing chamber can be recirculated to the homogenization chamber, a proportion can be recirculated within the ultrasonication loop, and a portion can exit the process. The proportions of these streams are not particularly limited, and the ratio of the stream recirculating back to the homogenization chamber relative to the proportion that recirculates within the ultrasonication loop relative to the proportion that exits the process can be about 1:1:1, 3:1:1, 2:1:1, 1:3:1, 1:2:1, 1:1:3, 1:1:3, 4:1:1, 1:4:1, 1:1:4, 5:1:1, 1:5:1, 1:1:5, and any other ration between.

At least one pump can be provided for circulation of the crude emulsion and ultrasonicated emulsions throughout the emulsion process. One or more valves can be provided within the system to control circulation flow rates and direction of the emulsion streams at various parts of the process. One or more heat exchangers can be included to control temperatures of the emulsion stream. The homogenization chamber can be a rotor stator homogenizer, a high-pressure homogenizer, a microfluidizer, an ultrasonic bath, and the like. In certain embodiments, an ultrasonic processing chamber comprises at least one ultrasonic probe, which can be made of titanium, ceramic, glass, alloys, piezoelectric high polymers, or a combination thereof. In certain embodiments, the at least one ultrasonic probe is an ultrasonic titanium probe. In certain embodiments, at least one ultrasonic processing chamber can comprise two or more probes, which can be arranged in series, parallel, or in combination within the chamber. In certain embodiments, at least one ultrasonic processing chamber can comprise at least one probe and at least one agitator to ensure emulsions within the chamber are exposed to the at least one probe uniformly. In certain embodiments, the at least one heat exchanger surrounds the ultrasonic processing chamber to cool down an ultrasonicated emulsion to about 40° C. to about 65° C. In certain embodiments, the recirculation loop comprises two heat exchangers, one being placed between the pump and the ultrasonic processing chamber to control the temperature of an emulsion before it flows into the ultrasonic processing chamber and the other surrounding the ultrasonic processing chamber to cool down an ultrasonicated emulsion. In certain embodiments, the recirculation loop comprises two valves, one controlling flow rates of an emulsion into the ultrasonic processing chamber and the other controlling flow rates of an emulsion out the ultrasonic processing chamber.

A valve outside the recirculation loop can be positioned between the ultrasonic processing chamber and the sedimentation/settling tank to allow an ultrasonicated emulsion containing no probe particles to flow from the ultrasonic processing chamber to the sedimentation/settling tank. An emulsion free of probe particles is then filtered through a filter.

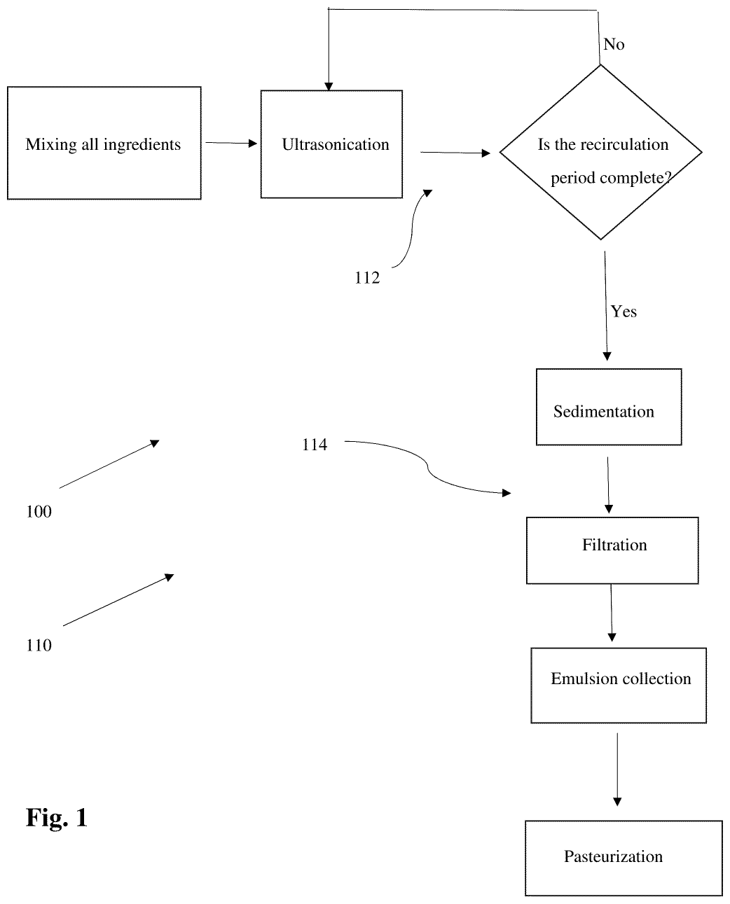

An example of the process is described in FIG. 1. All the ingredients in a composition (including water, one or more poor-water-soluble agents, one or more surfactants, and other applicable components described above) are homogenized to form a coarse emulsion which is then subjected to ultrasonication. The resultant ultrasonicated emulsion is recirculated via a ultrasonication/recirculation loop 112 until uniformed particles in the emulsion are formed (e.g., having an average particle size of between 0.1 μm and 20 μm). The resultant emulsion is then sedimented to remove probe particles followed by filtration to give a stable emulsion. The emulsion is further pasteurized.

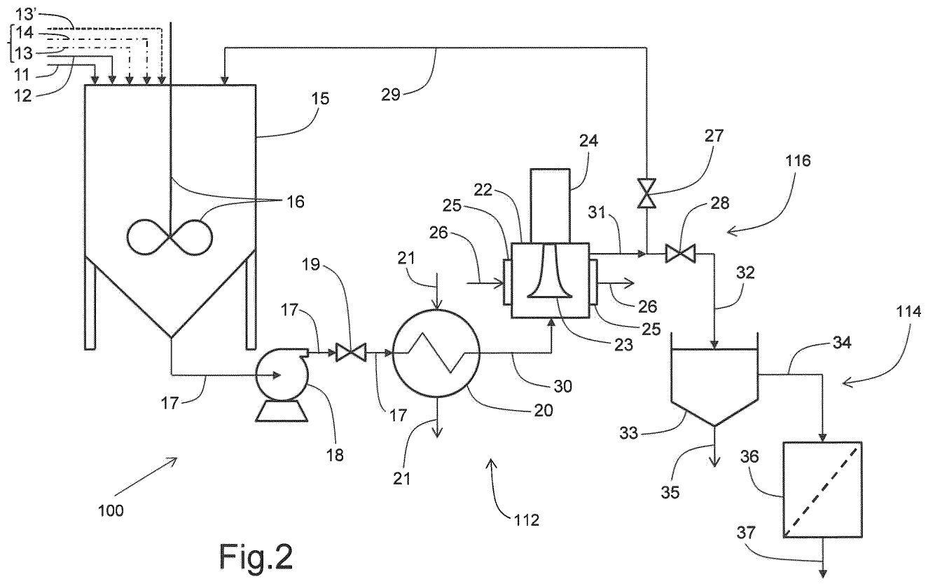

FIG. 2 shows an apparatus 100 and an exemplary process for producing an emulsion, in accordance with an embodiment of the present invention, from a coarse emulsion mixture typically comprising water 11, a poor-water-soluble agent 12, and at least one surfactant. In such an embodiment, the at least one surfactant can comprise a lecithin 13 (such as sunflower lecithin or the like) and polysorbate-80 14, and gum acacia 13′, although any surfactant can be used as set forth in the foregoing. The apparatus 100 can include a recirculation loop 112 for recirculating and processing coarse emulsion mixture 17 (or homogenized liquid, or composition, or recirculation stream) into a resulting liquid 32, and a treatment path 114 for treating a resulting liquid 34 to produce emulsion 37.

A valve member 116 can connect to, and between, the recirculation loop 112 and the treatment path 114. Valve member 116 can include a first valve 27 within the recirculation loop 112 to control circulation of the composition 31 within the recirculation loop 112, and a second valve 28 connecting the treatment path 114 to the recirculation loop 112, as further explained herein. Although not illustrated in the drawings, a person having ordinary skills in the art would readily recognize that the valve member could take different forms, with different combinations of different components, as, for example, in the form of a three-way valve to control the direction of the flow of the resulting liquid 32 within the loop 112 or towards the treatment path 114 and preferably a throttle valve to control the flowrate of the mixture 17, 29, 30, and 31 within the loop 112.

More specifically, in reference to FIG. 2, the invention is comprised of a number of unit operations in a specific sequence, with a specific input of materials. Input material streams 11, 12, 13-14, and/or 13′ are one or more poor-water-soluble agents and/or one or more surfactants. In certain embodiments, the one or more surfactants can comprise one or more of lecithin, polysorbate-80, and/or gum acacia. These three (3), four (4) or five (5) input streams 11, 12, 13-14, and/or 13′ enter into a homogenizer chamber 15 of the recirculation loop 112, where the liquid or mixture 17 is homogenized using the mixing impeller 16 with its shaft. When the proportions of input materials 11, 12, 13-14, and/or 13′ are successfully achieved (see hereinafter for more details), no more material is added into the homogenizer chamber 15. More specifically, the possible combinations can be of three (3) input streams (11, 12, 13′), four (4) input streams (11, 12, 13, 14), and five (5) input streams (11, 12, 13, 14, 13′), respectively.

The resulting homogenized liquid 17 is typically pumped through the process using a pump 18 or the like. The pump used is preferably a centrifugal pump 18. The target flowrate through the process can be controlled and maintained using a throttle valve 19 or the like typically located just downstream of the pump 18. The liquid of stream 17 then preferably passes through a heat exchanger 20, typically using cooling water as the heat transfer fluid flowing through stream 21.

The mixture 17, at controlled temperature and flowrate, then becomes the inlet stream 30 to an ultrasonic processing chamber 22 passes through the chamber 22 while contacting an oscillating titanium probe 23. The titanium probe 23 maintains its oscillating frequency and amplitude by a power supply and electrical convertor 24. The liquid 30 inside the ultrasonic processing chamber 22 is typically cooled using a heat exchanger 25 with cooling water 26 as the heat transfer fluid. The liquid inside the ultrasonic processing chamber 22 exits in effluent 31. The effluent 31 is recirculated back into the homogenizer chamber 15 through a recirculation stream 29. The homogenizer chamber 15 also serves the purpose of a recirculation tank. The recirculation occurs when a recirculation valve 27, located upstream of the recirculation stream 29, is opened, and when treatment valve 28, connecting with the treatment path 114, is closed.

After the predetermined recirculating period is complete, the recirculation valve 27 is closed, and the treatment valve 28 is opened to allow the resulting liquid 32, typically coming out of the ultrasonic processing chamber 32, to flow along the treatment path 114 into a sedimentation tank 33 (or settling tank), where titanium sediment 35 is removed and/or separated from the liquid 32. The clarified resulting liquid 34 from the sedimentation tank 33 then passes through 30 a filter member 36 to remove any microscopic impurities. The output filtrate 37 is then collected and further pasteurized to give a final emulsion product.

FIG. 2 also shows an exemplary apparatus that comprises a homogenization chamber 15, a pump 18, a first valve 19, a first heat exchanger 21, an ultrasonic processing chamber 22, an ultrasonic probe 23, a power supply and electric convertor 24, a second heat exchanger 25, a second valve 27, a third valve 28, a sedimentation/settling tank 33, and a filter 36. The method 110 comprises the steps of mixing a composition 17 of water 11, poor-water-soluble agent 12, and at least one surfactant, which can comprise one or more of lecithin additive 13 and polysorbate-80 14, and gum acacia 13′ to form the coarse emulsion mixture 17; and processing the coarse emulsion mixture 17 to get the poor-water-soluble agent in water emulsion 37.

In certain embodiments, the step of processing comprises the steps of recirculating the coarse emulsion mixture 17 within a recirculation loop 112 for a predetermined amount of time (or recirculating period) to get a resulting liquid 32; and treating the resulting liquid 32 to get the poor-water-soluble agent in water emulsion 37.

The step of recirculating can include the sub-steps of homogenizing the mixture 17, pumping the mixture 17 through (or within) the recirculation loop 112 (using the pump 18), controlling the flowrate of the mixture 17 into the recirculation loop 112 (using the throttle valve 19), controlling the temperature of the mixture 17 before entering into the ultrasonic processing chamber 22 (which can be via cooling the temperature of the mixture 17 down via the heat exchanger 20), and ultrasonicating (or ultrasonic processing of) the mixture 30 to break down the large emulsion particles present in the mixture into much smaller emulsion particles, which aid in increasing the solubility of the poor-water-soluble agents as described herein. More specifically, the coarse emulsion 17 can flow through the ultrasonic processing chamber 22 at a constant flowrate, exposing the coarse emulsion to the energy produced by the rapidly oscillating titanium probe 23. The energy delivered by the probe 23 creates areas of drastic pressure differences in the liquid 17, which generates cavitation bubbles that collapse and result in intense shock waves travelling typically faster than the speed of sound. These shock waves violently split larger emulsion particles, thereby forming much smaller emulsion particles that are much more stable. This phenomenon typically creates heat, and the liquid 30 inside the ultrasonic processing chamber 22 is cooled by using the heat exchanger 25 with cooling water 26 flowing therethrough.

The step of treating typically includes the sub-steps of separating titanium sediments 35 (from the titanium probe 23) from the resulting liquid 32 to get a clarified resulting liquid 34 (with the sediment 35 being recuperated from the sedimentation tank 33), and filtrating the clarified resulting liquid 34, via the filter member 36, to remove remaining impurities therefrom, resulting in the stabilized emulsion mixture comprising a water-miscible aqueous solution comprising a poor-water-soluble agent 37 at a concentration unexpectedly greater than that achieved by the prior art. The emulsion can be pasteurized, which can be employed to reduce the likelihood of the growth of any microbial or other organic lifeforms.

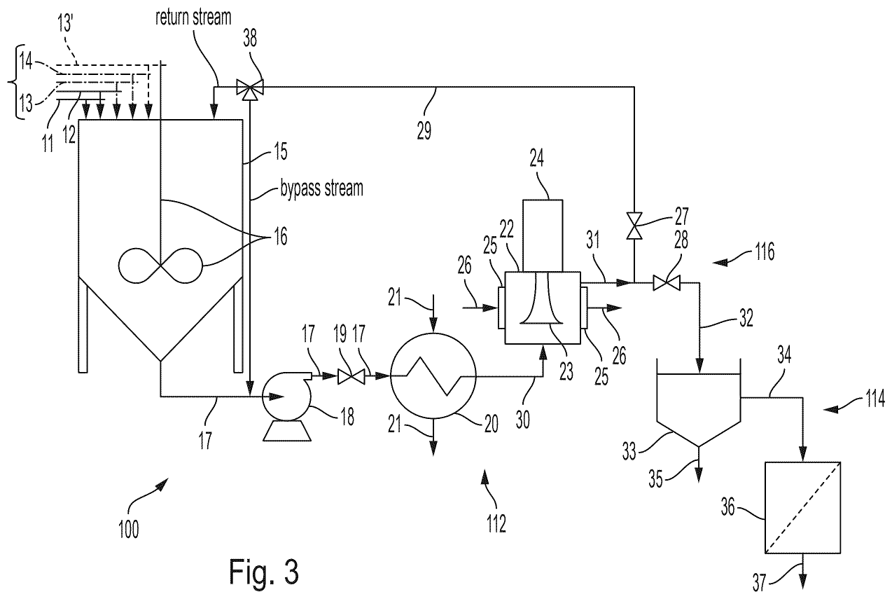

FIGS. 3-5 display a non-limiting set of exemplary embodiments of different configurations of process flows envisaged by the disclosure contained herein.

FIG. 3 varies from FIG. 2 in terms of the splits of the product stream 31 existing the ultrasonic processing chamber. Specifically, FIG. 3 shows the product stream 31 exiting the ultrasonic processing chamber can be split according to any ratio set forth herein (e.g., 1:1, 2:1, 3:1, 4:1, 5:1, 6:1, 7:1, 8:1, 9:1, 10:1, and any ratio therebetween) such that, e.g. 10%, of the product stream exits the ultrasonic processing chamber and is directed to the filter member and/or settling chamber, and the remainder of the product stream, e.g. the remaining 90%, is circulated back to the homogenizer and/or ultrasonication loop. The product stream 29 can be split via valve 38 between the homogenization chamber and the bypass stream that bypasses the homogenization chamber and flows back to the recirculation loop. The split of the product stream and the bypass stream is not particularly limited and can be in accord with any volume ratio set forth herein (e.g., 1:1, 2:1, 3:1, 4:1, 5:1, 6:1, 7:1, 8:1, 9:1, 10:1, and any ratio therebetween). In some embodiments, the entire product stream can bypass the homogenization chamber and circulate within the recirculation loop.

An apparatus used in the preparation of a composition described herein can comprise more than one ultrasonic processing chamber arranged in series or parallel or in a manner that would be immediately envisaged by the skilled artisan in view of the disclosure contained herein. These multiple ultrasonic processing chambers can allow for increased flow rates and reduced processing time, increased exposure time, increased residence time, and/or the ability to process a larger amount of aqueous solution per batch. In certain embodiments, the product stream entering each ultrasonic processing chamber can be in equal amount or in different amounts.

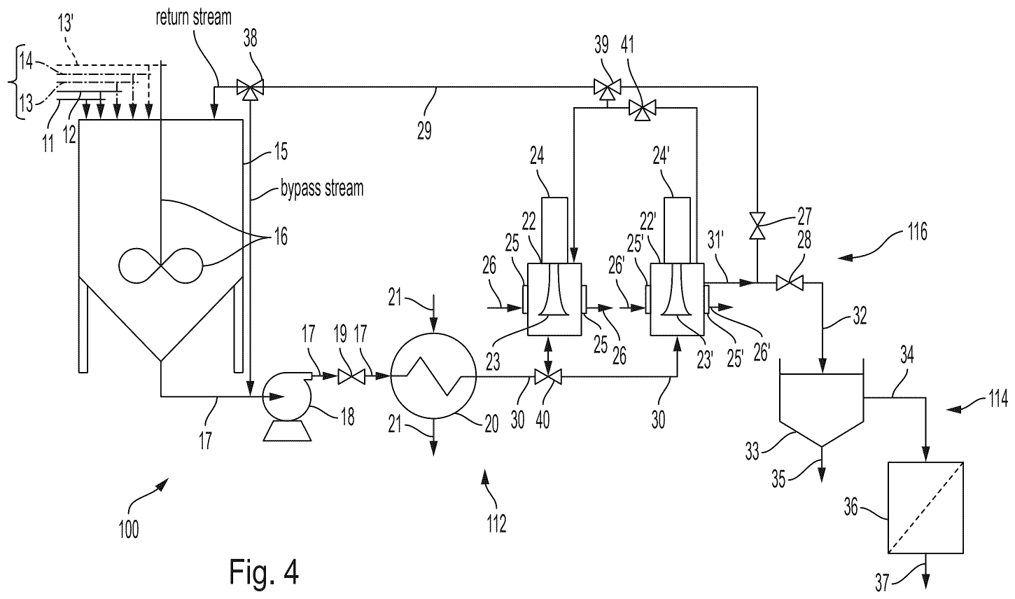

FIG. 4 shows an exemplary configuration comprising two ultrasonic processing chambers 22 and 22′ arranged in parallel. Chamber 22′, like chamber 22, contains an ultrasonic probe 23′ powered by an electric convertor 24 and is surrounded by a heat exchanger 25′, which can have water 26′ flowing through to cool down ultrasonicated liquid 30 within chamber 22′. The amount of liquid 30 entering chambers 22 and 22′ can be in a ratio of 1:1, 2:1, 3:1, 4:1, 5:1, 6:1, 7:1, 8:1, 9:1, 10:1, and any ratio therebetween. Valves 27, 39, and 40 can be used to control the amount and flow rate of liquid from each chamber into the recirculation loop. Alternatively, ultrasonic processing chambers 22 and 22′ can form an ultrasonication loop where liquid 30 can be transferred from one chamber (e.g., chamber 22′) to the other chamber (e.g. chamber 22).

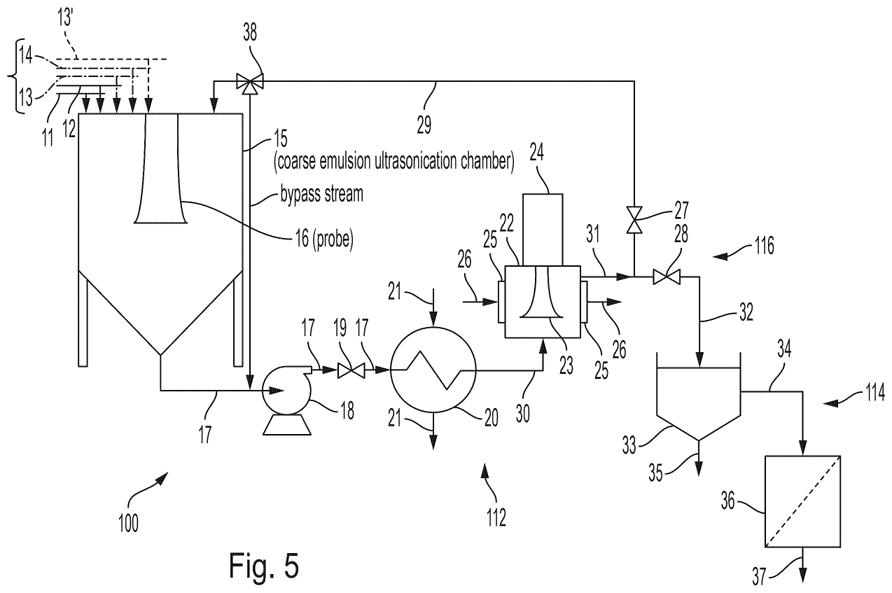

FIG. 5. shows an exemplary arrangement where an ultrasonic processing chamber 15 containing a probe 16 is used to replace the homogenized chamber shown in FIG. 3 to prepare a coarse emulsion. The ultrasonic processing chamber 15 can further contain one or more agitators to homogenize a mixture of ingredients in a composition.

The terminology used herein is for the purpose of describing particular embodiments only and is not intended to be limiting. As used in the Specification and the appended claims, the singular forms “a”, “an” and “the” include plural referents unless the context clearly dictates otherwise.

As set forth herein, all numerical values should be treated as being preceded and therefore modified by the term “about,” in accordance with applicable law and the understanding ascribed to those terms by the skilled artisan in view of the context in which the term is used and the disclosure contained herein.

The term “composition” and the like can refer to preparations which are in such a form as to permit the biological activity of the active ingredients to be effective, and therefore may be administered to a subject for therapeutic use along with dietary and/or nutritional supplement use. The term “emulsion” can refer to a mixture of two or more liquids that are normally immiscible owing to liquid-liquid separation. The term “homogenize” can refer to any processes used to make a mixture of two mutually non-soluble liquids the same throughout by turning one of the liquids into a state consisting of extremely small particles distributed uniformly throughout the other liquid. In some instances, the term “composition” and “emulsion” are interchangeable. The meaning of these terms will be clear to the skilled artisan based upon the context in which they are used.

As provided herein, the disclosure of a “ratio” of compounds and compositions corresponds to a ratio provided in terms of weight mass of the components present in the ratio.

Although the present disclosure describes methods and processes with steps in a certain order, one or more steps of the methods and processes may be omitted or altered as appropriate. One or more steps may take place in an order other than that in which they are described, as appropriate.

The present disclosure may be embodied in other specific forms without departing from the subject matter of the claims. The described example embodiments are to be considered in all respects as being only illustrative and not restrictive. Selected features from one or more of the above-described embodiments may be combined to create alternative embodiments not explicitly described, features suitable for such combinations being understood within the scope of this disclosure.

All values and sub-ranges within disclosed ranges are also disclosed. Also, although the systems, devices and processes disclosed and shown herein may comprise a specific number of elements/components, the systems, devices and assemblies could be modified to include additional or fewer of such elements/components. For example, although any of the elements/components disclosed may be referenced as being singular, the embodiments disclosed herein could be modified to include a plurality of such elements/components. The subject matter described herein intends to cover and embrace all suitable changes in technology.

### EXAMPLES

### Example 1: A Flavor-Oil-Containing Emulsion

A water-miscible emulsion containing about 10 wt % orange flavor oil, about 3.333 wt % MCT oil, and about 7 wt % quillija extract was prepared by a process described in FIGS. 1 and 2. For at least two years at room temperature, the emulsion did not change its appearance, i.e., no water-oil separation. The emulsion (10 mg) was added to a drink product (e.g., purified water, 490 mg) to give a clear orange-flavored water solution having about 0.2 wt % of orange flavor oil. The resulting water-oil solution was stable at room temperature without water-oil separation for at least 2 years.

### Example 2: An Algal Oil-Containing Emulsion

A water-miscible emulsion containing about 10 wt % algal oil, about 3 wt % sunflower lecithin, and about 2 wt % quillija extract was prepared by a process described in FIGS. 1 and 2. For at least two years at room temperature, the emulsion did not show no water-oil separation. The emulsion (10 mL) was added to water (330 mL) to give a clear water-oil solution that was stable without water-oil separation at room temperature for 2 years.

### Example 3: Stability of a Coarse Hemp Oil Emulsion and a Hemp-Oil-Containing Water-Miscible Emulsion

400 mL of a coarse hemp oil emulsion containing about 10 wt % hemp oil, about 3 wt % sunflower lecithin, and about 2 wt % quillija extract was prepared in accordance with FIGS. 1 and 2 described above. 100 mL of the coarse emulsion was taken out and the remaining 300 mL was ultrasonicated via a recirculation process as described in FIGS. 1 and 2 to give a water-miscible emulsion. Both emulsions were placed at room temperature to evaluate their stability. It was observed that in the coarse emulsion, hemp oil was separated from water during a course of 2 years while in the water-miscible emulsion, hemp oil was not separated from water. The result demonstrates that the recirculation process increases the stability of a water-oil emulsion.

### Example 4: Particle Sizes of a Water-Miscible Hemp Oil Emulsion

Particle sizes in a water-miscible emulsion containing about 10 wt % of hemp oil was determined using Beckman Coulter LS Particle Size Analyzer. The average particle size was about 2.310 μm, less than 10% particles had a size of about 0.685 μm, and 90% particles had a size of about 12.62 μm.

### Example 5: Bioavailability Study

A comparative study will be conducted to determine the bioavailability of a composition described herein (“the Composition”) as compared to the bioavailability of a reference composition (“the Reference Composition”). An example of one such study that may be employed, a water-miscible emulsion containing about 10 wt % of hemp oil described herein will be compared to a reference composition prepared by mixing pure hemp oil (1 g) with water (9 g) in terms of their bioavailability.

As permeability is a key determinant of bioavailability, the bioavailability will be evaluated by an in vitro permeability study using Caco-2 permeability assay.

### Cell Line and Culture Medium

In 96 well microtiter plates, Caco-2 (Colon carcinoma) cells will be cultured with a medium containing 10% inactivated Fetal Bovine Serum, penicillin (100 IU/ml), streptomycin (100 g/ml), and amphotericin B (5 g/ml) at 37° C. in a humidified environment of 5% CO2. Trypsin Phosphate Versene Glucose (0.2% trypsin, 0.5% PVP, 0.02% EDTA, and 0.05% glucose in PBS) will be used to separate the cells.

### Cytotoxicity Study

Both compositions will be initially examined for their in vitro cytotoxicity against Caco-2 cells by using the 3-(4,5-dimethylthiazol-2-yl)-2,5-diphenyl-2H-tetrazolium bromide (MTT) assay in semi-confluent monolayer cultures. This study will be conducted to ensure the accuracy of cell permeability results; namely, increased cell permeability is not caused by damages of cells. The cells will be treated with a vehicle, the Composition, or the Reference Composition at various concentrations, then incubated at 37° C. under a 5% CO2 environment in wells of a plate for 72 hours. After that, each of the resultant solutions will be carefully removed from the wells, and 100 μl of MTT in a phosphate-buffered saline will be added to each well. The cells will be incubated for another three hours at 37° C. under a 5% CO2 environment to give formazan. The supernatant will be then removed. Dimethyl sulfoxide (DMSO) will be added to solubilize the formazan. At a wavelength of 540 nm, the absorbances of the formazan will be determined using a microplate reader to give dose-response curves. The common toxicity criteria 50 (CTC50; defined as the concentration of a substance killing 50% of total cells/inhibit 50% of the cell growth) values will be calculated from the curves. The cytotoxicity will be given as a percentage compared to that of the cells treated with the vehicle.

### Caco-2 Permeability Study

Caco-2 cells between passages 30 and 35 will be seeded on polyester membrane inserts (113.1 mm, 0.4 μm pore size) at a density of 100,000 cells per insert and cultured at 37° C. for 25 days, applying culture medium changes three times a week to allow the formation of a confluent monolayer. The integrity of the monolayer will be assessed using Lucifer Yellow (LY) dye to determine LY apparent permeability coefficient (LY Papp) by the formula below:

$${P}_{app} = \frac{\frac{dQ}{dT}}{( A ) ( Cd )}$$

dQ/dT is the rate of appearance of LY(μg/sec) in the basolateral layer, A is an area of the semi-permeable membrane, and Cd is initial concentration of LY in the apical layer.

The monolayer having LY Papp less than 0.2×10⁻⁶ cm/sec will be used for the permeability study. To mimic the in vivo conditions, the pH value of the apical layer will be adjusted to 6.0 and the pH value of the basolateral layer will be adjusted to 7.4 by using a balanced salt solution. The DMSO solution of the Composition and the DMSO solution of the Reference Composition will be diluted with the balanced salt solution to give a concentration of 62.5 μg/ml. Each of the resultant solutions will be added to the apical layer individually. The cells will be then incubated for 2 hours at 37° C. within an orbital shaker spinning at 100 rpm. Before the incubation and at hour 2 of the incubation, 100 μl of each solution will be collected from both the apical layer and the basolateral layer and will be substituted with equal volumes of the balanced salt solution. Each solution will be analyzed by HPLC as compared to the standard. The following formula will be used to calculate the permeability coefficient (PBAapp):

$${PBA}_{app} = \frac{Δ Q}{Δ T} \times \frac{1}{A * {C}_{0} * 60}$$

where 66 Q/ΔT is the steady state flux (mol/sec), A is the surface area of the filter (mm²), and C₀ is the initial concentration of the Composition/Reference Composition applied in the apical layer (mol/ml).

1. A water-miscible composition comprising one or more poor-water-soluble agents and one or more surfactants.

2. The water-miscible composition of claim 1, wherein the poor-water-soluble agents are lipids.

3. The water-miscible composition of claim 2, wherein a total weight percentage of the one or more lipids is present at least about 5 wt %.

4. The water-miscible composition of claim 3, wherein a total weight percentage of the one or more lipids is present at about 10 wt % to about 50 wt %.

5. The water-miscible composition of claim 2, wherein the one or more lipids comprise fat-soluble vitamins, hemp seed oil, curcumin, black seed oil, flax seed oil, hops oil, algal oil, oregano oil, MCT oil, omega 3, omega 6, omega 9, a phospholipid, or any combination thereof.

6. The water-miscible composition of claim 2, wherein a total weight percentage of the one or more surfactants are present at most about 60 wt %.

7. The water-miscible composition of claim 6, wherein a total weight percentage of the one or more surfactants are present at about 0.6 wt % to about 60 wt %.

8. The water-miscible composition of claim 2, wherein the one or more surfactants comprise lecithin, quillaia extract, gum acacia, a polysorbate, or any combination thereof.

9. The water-miscible composition of claim 8, wherein the lecithin is sunflower lecithin.

10. The water-miscible composition of claim 8, wherein the polysorbate is polysorbate 80.

11. The water-miscible composition of claim 2, wherein each of the one or more surfactants is present at about 0.2 wt % to about 20 wt %.

12. The water-miscible composition of claim 2, wherein the composition comprises a first surfactant, a second surfactant, and a third surfactant.

13. The water-miscible composition of claim 12, wherein a weight percentage of the first surfactants is present at about 1 wt % to about 3 wt %, a weight percentage of the second surfactants is present at about 2 wt % to about 4 wt %, and a weight percentage of the third surfactants is present at about 5 wt % to about 8 wt %.

14. The water-miscible composition of claim 1, wherein the poor-water-soluble agents are low-water-soluble agents.

15. The water-miscible composition of claim 14, wherein a total weight percentage of the one or more low-water-soluble agents is present at least about 5 wt %.

16. The water-miscible composition of claim 14, wherein the low-water-soluble agents have improved water solubility.

17. The water-miscible composition of claim 1, wherein the composition has a pH value of about 5.5.

18. A method of preparing a water-miscible composition comprising:
(a) mixing one or more surfactants and water;
(b) adding one or more poor-water-soluble agents to the resultant mixture to yield an emulsion;
(c) homogenizing the resultant emulsion;
(d) ultrasonicating the homogenized emulsion to give a water-miscible composition;
(e) removing impurities from the water-miscible composition; and
(f) pasteurizing the composition.

19. The method of claim 18, wherein step (d) further comprises ultrasonicating the emulsion at least three time by circulating the emulsion through a loop.

20. The method of claim 18, wherein the emulsion is ultrasonicated at a frequency of about 10,000 to about 40,000 Hz and at an amplitude of about 0.01 to about 1 mm.

21. The method of claim 18, wherein an acid or a base can be added in step (a) or step (b).

22. An apparatus used for preparing a water-miscible composition comprising a homogenization tank, a pump, at least two valves, at least one heat exchanger, an ultrasonic processing chamber, a sedimentation/settling tank, and a filter.

23. The apparatus of claim 22, wherein one of the at least two valves is placed between the ultrasonic processing chamber the sedimentation/settling tank.

24. The apparatus of claim 22, wherein the at least one heat exchanger surrounds the ultrasonic processing chamber.
